(19) United States
(12) Patent Application Publication
(10) Pub. No.: US 2020/0163438 A1
(43) Pub. Date: May 28, 2020

(54) Multiple Compartment Package

(71) Applicant: Lynda Tanya Correa Peralta, Washington, DC (US)

(72) Inventor: Lynda Tanya Correa Peralta, Washington, DC (US)

(21) Appl. No.: 16/778,254

(22) Filed: Jan. 31, 2020

Related U.S. Application Data
(62) Division of application No. 15/662,470, filed on Jul. 28, 2017.
(60) Provisional application No. 62/367,799, filed on Jul. 28, 2016.

(51) Int. Cl. A45D 40/00; B65D 75/58; A45D 40/26; A45D 40/24

(52) U.S. Cl. CPC A45D 40/0087; B65D 75/5855; A61M 35/006; A45D 40/24; A45D 40/262

(57) ABSTRACT

A disposable travel package for health and/or beauty products, including but not limited to makeup, cosmetics, skincare, and related accessories has multiple compartments that are formed between two sheets of plastic. The sheets can be scored to create an easy opening so that the user can tear open the package in a single operation to open all the compartments at one time.

### RELATED APPLICATIONS

This application is a Divisional of U.S. Utility application Ser. No. 15/662,470, filed on Jul. 28, 2017, which in turn claims the benefit of U.S. Provisional Application No. 62/367,799, filed Jul. 28, 2016, the entire contents of which are incorporated herein by reference.

### FIELD OF THE TECHNOLOGY

The present invention relates to a multiple compartment package. More specifically, the present invention relates to a travel package for health and/or beauty products, including but not limited to makeup, cosmetics, skincare, and related accessories.

### BACKGROUND

Various packages have been designed for retaining consumer products, such as cosmetics and the like, as shown in U.S. Pat. Nos. 8,003,116, 7,641,409, 6,213,303, 6,006,916, 5,879,769, and 4,889,228. However, those packages can be awkward to use and are not suitable for kits having multiple consumer products that are utilized together.

### SUMMARY

A disposable package for health and/or beauty products has multiple compartments that are formed between two sheets of plastic. The sheets can be scored to create an easy opening so that the user can tear open the package in a single operation to open all the compartments at one time. The health and/or beauty products include but are not limited to makeup, cosmetics, skincare, and related accessories.

### BRIEF DESCRIPTION OF THE DRAWINGS

FIG. 1 is a top view of the package according to the invention;

FIG. 2 is a side cutaway view of the package taken along line A-A of FIG. 1;

FIG. 3 shows an alternative configuration for the package;

FIG. 4 shows another alternative configuration for the package;

FIG. 5 shows a single compartment having guide members and a spout;

FIG. 6 shows another illustrative embodiment of the package;

FIG. 7 shows a zipper for use with the package;

FIG. 8 shows a press seal for use with the package;

FIG. 9 shows another illustrative embodiment of the package; and

FIGS. 10(a)-(c) show other illustrative embodiments of the package.

### DETAILED DESCRIPTION

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in similar manner to accomplish a similar purpose. Several preferred embodiments of the invention are described for illustrative purposes, it being understood that the invention may be embodied in other forms not specifically shown in the drawings.

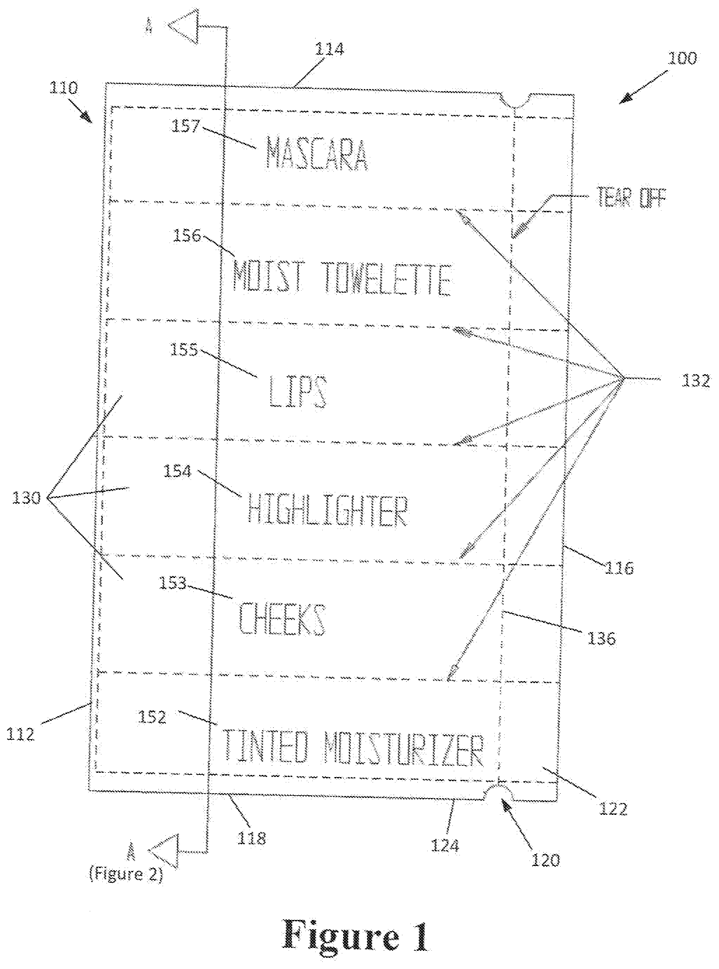

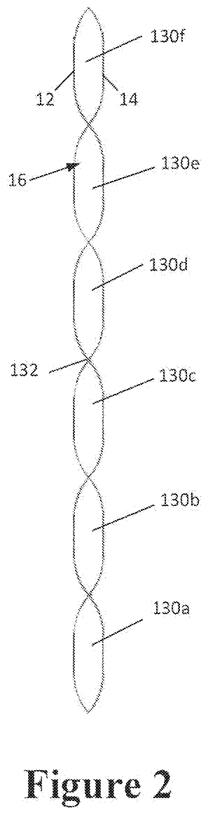

Turning to the drawings, FIGS. 1, 2 show the package 100 in one illustrative, non-limiting example of the present invention. The package 100 has a body 110 formed by a first sheet 12 and a second sheet 14. The first and second sheets 12, 14 can be made of plastic material, though any suitable flexible material can be utilized such as but not limited to Mylar, polyester, resin Polyethylene Terephthalate. The first sheet 12 forms a base layer and the second sheet 14 forms a top layer, and defines an interior space 16 there between.

The first and second sheets 12, 14 are in parallel planes and aligned with one another. The sheets 12, 14 are coupled together along the outer periphery of the sheets 12, 14 to form a single unitary body 110. For instance, the sheets 12, 14 can be heat sealed to form a sealed outer periphery that prevents liquid and dry materials from escaping. The unitary body 110 can have any suitable size and shape, though in one exemplary embodiment the body 110 is rectangular in shape and has a bottom 112, a first side 114, a top 116 opposite the bottom 112, and a second side 118 opposite the first side.

A plurality of compartments 130 are formed in the body 110 by forming a plurality of dividing walls 132. For example, the dividing walls 132 can be formed by heat sealing the first and second sheets 12, 14 to each other. As shown, the dividing walls 132 can be at positions that are parallel to the sides 114, 118 to form rectangular compartments 130. Thus, the compartments 130 are elongated and arranged serially, with each compartment 130 directly adjacent to a neighboring compartment 130. Each compartment 130 is defined by the bottom 112, top, 116, as well as by two dividing walls 132 or one side 114, 118 and one dividing wall 132. Thus, the compartments 130 extend from the bottom 112 to the top 116. Each compartment is directly adjacent one or two other compartments 130, whereby the compartments 130 share one or two adjoining dividing walls 132 with the immediately adjacent neighboring compartment 130.

It is noted that the compartments 130 are shown as being uniform in size and shape. And, the compartments 130 extend parallel to each other, whereby each has a length defining a longitudinal axis that is parallel to the longitudinal axis of the other compartments 130 and perpendicular to the longitudinal axis of the body 110, and with the tear line 136 extending along the longitudinal axis to cross the width of each compartment at one end. However, it will be appreciated that the compartments 130 can have any suitable size and shape, and the compartments 130 need not all be the same size and/or shape. In addition, the compartments 130 need not be adjacent one another or adjoining, but can be separated on the body 110. And the compartments 130 need not be parallel to the longitudinal axis of the body 110, but can be configured with any arrangement such as perpendicular to the longitudinal axis of the body 110. And the tear line 136 need not extend along the longitudinal axis, but can extend across any dimension or portion of the compartments.

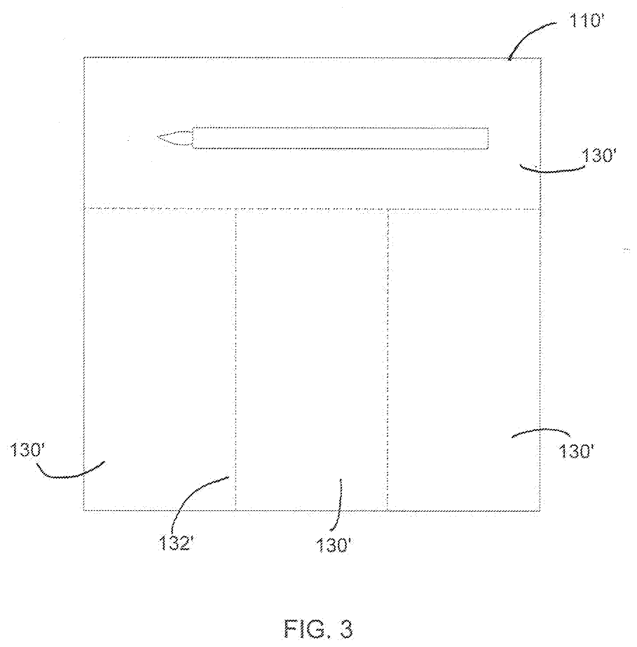

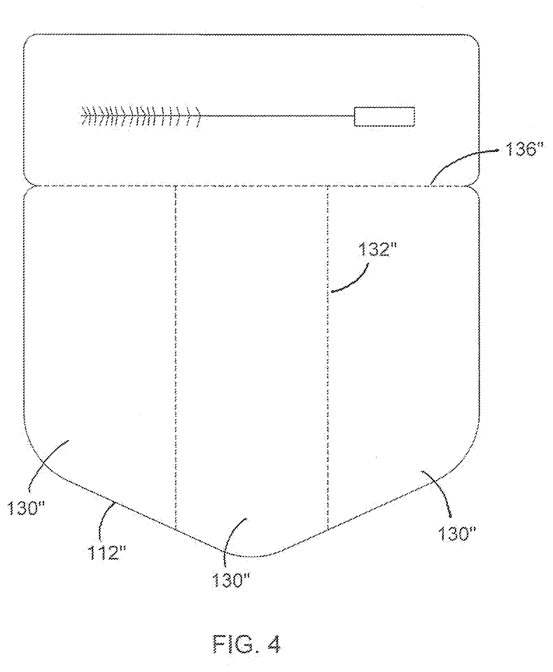

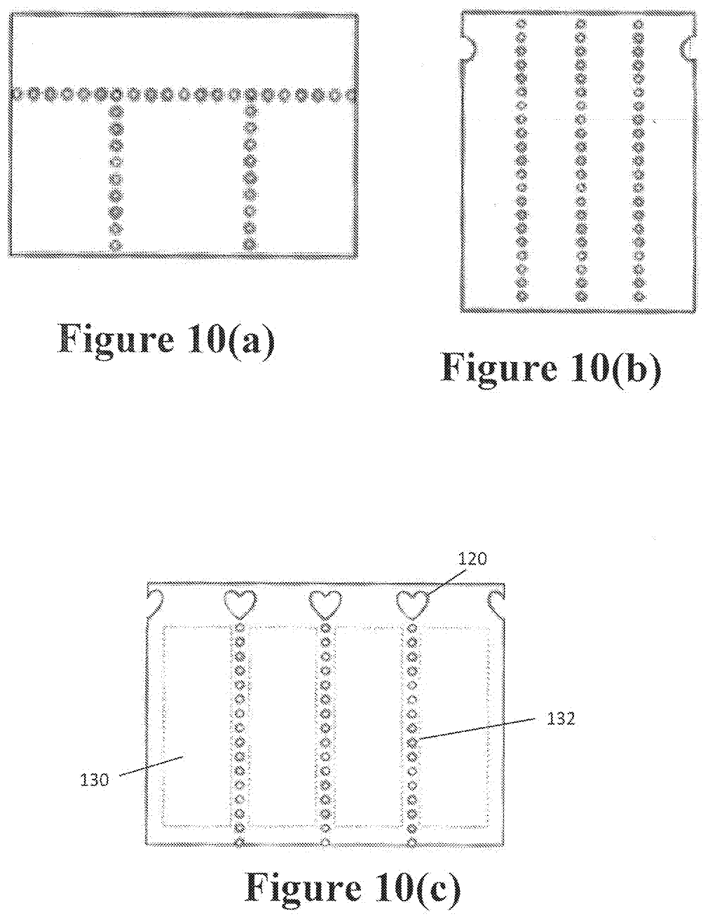

For example, non-limiting embodiments of the invention are shown in FIGS. 3, 4, 10(a), 10(b). Here, the compartments 130′, 130″ have different shapes and sizes. As illustrated in FIGS. 3 and 10(a), there can be three compartments 130′ that are substantially parallel to each other, with a larger middle compartment 130′. And there is a fourth compartment 130′ that is substantially perpendicular to the other compartments 130′, and is longer and narrower than the other compartments 130′. As further shown in FIG. 4, the bottom 112″ can be curved, so that the compartments 130″ are curved at the bottom 112″. As shown in FIG. 10(b), the compartments can be elongated and parallel to one another and have a longitudinal axis that is parallel to the longitudinal axis of the entire package, with notches toward the top end of the left and right sides of the package so a user can open all of the compartments by tearing it open from one notch to the other.

As shown in FIG. 2, the package 100 has multiple compartments 130a-130f formed by the space 16 located between the first sheet 12 and the second sheet 14. Referring to FIG. 1, a product 152-157 (such as a material (e.g., makeup, liner), liquid or device (e.g., applicator)) is received in each compartment 130, so that each compartment 130 retains a single product 152-157. The products 152-157 can each be the same or different, and can be health and/or beauty products or items, including but not limited to makeup, cosmetics, skincare, and related accessories. For instance, a first compartment 130a can have a tinted moisturizer product 152, a second compartment 130b can have a cheek makeup product 153, a third compartment 130c can have a highlighter 154, a fourth compartment 130d can have lipstick product 155, a fifth compartment 130e can have a moist towelette product 156, and a sixth compartment 130f can have a mascara product 157. Since the products can be different, the compartments 130 can be differently sized or shaped to accommodate the different sized and shaped products.

In one exemplary embodiment, the first sheet 12 can be opaque and the second sheet 14 can be transparent so that the user can see the contents (i.e., product) inside of the compartments 130. However, both sheets 12, 14 can be transparent, or both sheets 12, 14 can be opaque, or both sheets 12, 14 can have a design that is partially opaque and/or partially transparent.

The body 110 also includes a mechanism for opening the compartments 130 in a single operation by the user, where the user can tear open all the compartments 130 at one time. In the embodiment of FIG. 1, the opening mechanism is shown as a notch 120 that is provided at one or both sides 114, 118 of the body 110. The notches 120 define a tear line 136 that extends from the notch 120 on the first side 114 to the notch 120 on the second side 118. The tear line 136 separate the body 110 into a top portion 122 and bottom portion 124 of the body 110. The user can use either one of the notches 120 to pull open the body 110 by exerting opposite forces at the notch 120, such as by exerting opposite forces at the top portion 122 of the body 110 and at the bottom portion 124 of the body 110 just at or below the notches 120. That force will cause the sheets 12, 14 to tear open along the tear line 136. Optionally, the tear line 136 can be a weakened area (such as being scored or the like) that is still liquid and material proof, so that the tear occurs evenly along the tear line 136. The notches 120 can also be positioned to enable the user to tear open the package. The notches 120 can be provided with or without a tear line 136, and the notches 120 and/or tear line 136 can be positioned within an end of the compartment to pen the compartment or can be positioned outside a compartment (such as within a dividing wall 132) to separate compartments.

Once the user tears open the package 100, all of the compartments 130 become accessible to the user at the same time as a result of a single operation by the user (here, tearing the package). Accordingly, the user can access the moisturizer product 152 in the first compartment 130a at the same time as the cheek makeup product 153 in the second compartment 130b. In this way, the package 100 is especially useful for retaining multiple products 152-157 that form a kit having different products that are useable together. In the example of FIG. 1 for instance, the kit includes six compartments 130a-f that all relate to the same application type or product type (here, beauty products), though the products themselves differ. Thus, two or more (or all) of the product can be used at the same time for the same application type (or for more than one application type).

As shown in FIG. 3, the divider wall 132′ can be a thickened area and can have circular openings that extend through the body 110′ along a heat seal so the user can see where the compartments are separated and/or so that the compartments can be separated from one another by tearing along those openings without opening the compartment or discharging its contents. And as shown in FIG. 4, the divider wall 132″ can be a thickened area and a weakened score line 136″ can extend through the center of the heat seal to enable the compartments to be separated. The score line 136″ can also be at one edge of the heat seal so that a compartment can be opened by being torn open.

In FIG. 1, for example, the user would open all the compartments at one time and use two or more of the products 152-157, such as when preparing to leave the house for work, an event or an appointment. The user might use the lipstick 155 and the highlighter 154 at that time, or all of the products 152-157. And some of the products can directly be used together, such as cheek makeup 153 in one compartment and a cheek applicator in another compartment. Although six compartments 130 are shown, more or fewer compartments 130 can be provided within the spirit and scope of the invention. And although the application type here relates to health and/or beauty products, the products can related to any suitable product type or application type. And although the user preferably opens all the compartments in a single user operation (i.e., a single operation by the user), it should be noted that the user can open fewer than all of the compartments, such as one or more.

The package 100 is particularly useful for travel. The amount of product in each compartment 130 can be enough for a single use, or for several uses (such as for 2-3 days or even a week or more). Once the package 100 is opened and the product is at least partially used, the package 100 can be discarded together with any remaining product.

To prepare the package 100, a first step is to couple the first sheet 12 to the second sheet 14, such as by heat sealing the edges of the sheets 12, 14 to one another. In a second step, the compartments 130 are formed. The second step can be performed at the same time, before or after the first step. The second step can be done by heat sealing the divider walls 132. In a third step, the product 152-157 is placed in each of the compartments 130. This can be done by placing the product between the sheets 12, 14 before conducting steps 1 and/or 2. Or, the sides 114, 118, bottom 112, and divider walls 132 can be heat sealed to partially form open-ended compartments 130 (i.e., compartments with an opened top). The product 152-157 can then be filled into the open top of the partially-formed compartments 130. The top 116 can then be closed, such as by heat sealing, to form the closed compartments 130 with the product contained therein.

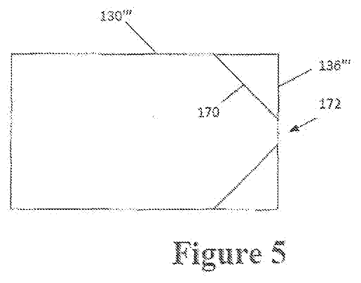

Turning to FIG. 5, an optional feature of the invention is shown. Here, a single compartment 130′″ is shown. A score line 136′″ is provided along one end of the compartment 130′″. In addition, the compartment 130′″ has guide walls 170 that are tapered inward to form a narrowed nozzle or spout 172 at the end of the compartment that is scored. The guide wall 170 can be formed, for example, by heat sealing the body of the package. Accordingly, material and product do not extend beyond the guide walls 170, and the guide wall 170 direct any material or product in the compartment 130′″ out through the opening at the spout 172. The spout 172 is opened when the user opens the compartment by the tear line 136′″.

In one illustrative, non-limiting embodiment, the body 110 of FIGS. 1-2 with three compartments 130 can have a length of about 3 inches and a width of about 1.8 inches, so that each compartment 130 is 3 inches in length and about 0.5 inches in width. And FIGS. 3, 4 can have a length of about 3.5 inches and a width of about 3 inches, so each of the three side-by-side compartments has a width of 1 inch and length of about 3 inches. However, any suitable sizes and shapes can be provided within the spirit and scope of the invention.

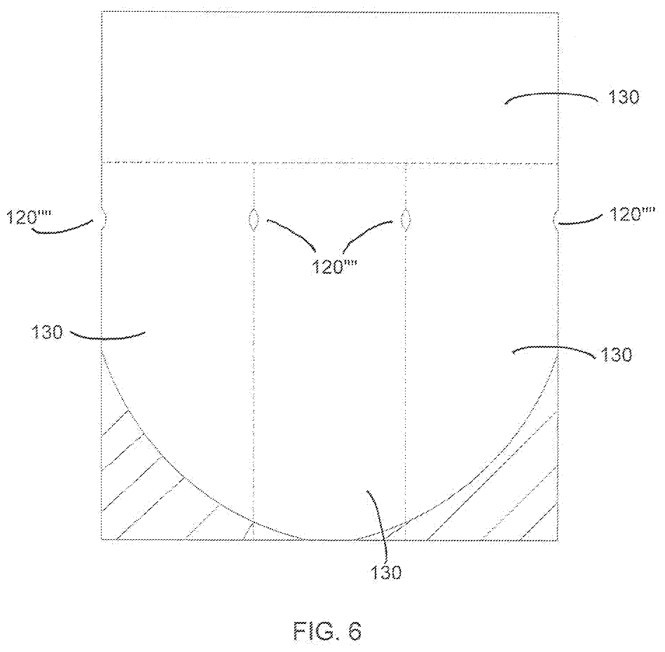

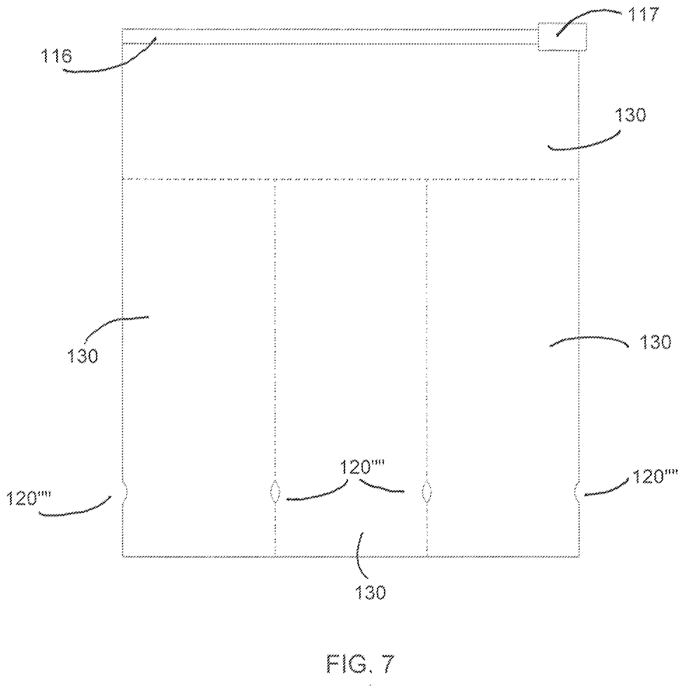

FIGS. 6-10 show other non-limiting illustrative embodiment of the invention and show various alignments of the compartments parallel and/or perpendicular to each other. In addition, the package can have any suitable opening mechanism to open the various compartments other than a tear line. For example referring to FIG. 6, the package contains notches 120″″ for the user to open the various compartments. FIG. 7 shows the package having a resealable seal with a zipper 117 for an opening mechanism. The zipper 117 is positioned along a top edge 116 of the package. The zipper 117 can be a press-type zipper that extends along a sealing member or track on each of the plastic sheets so that the zipper can resealingly press the two sealing members together to seal and unseal the sealing members when the zipper is moved from one side to the other. As shown, the zipper extends across one compartment 130 so that the user can open and close that compartment 130 to insert and remove items. However, the zipper 117 can extend across one or more or all of the compartments 130 to open them at the same time.

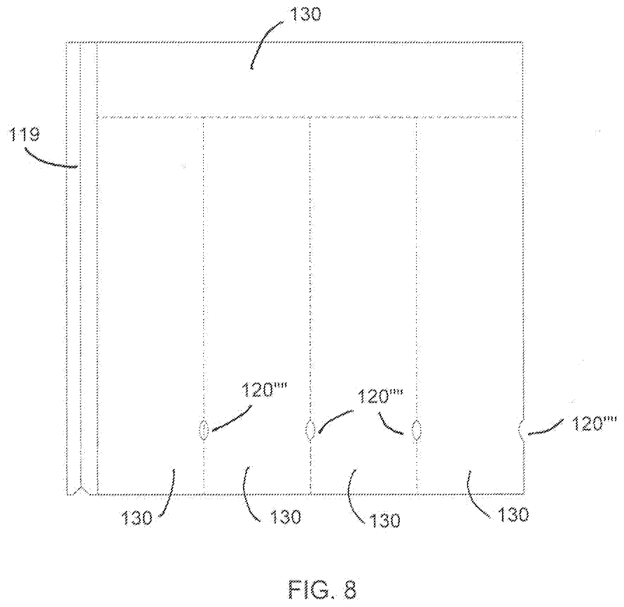

FIG. 8 shows a resealable seal 119 for an opening mechanism. A sealing member is positioned on each of the plastic sheets, but without a zipper. The plastic sheets are pressed together to join or seal the sealing members, and the user can open the package compartments by pulling the sealing members apart. As with the opening mechanism of FIG. 7, the releasable seal 119 can be aligned along one compartment 130, as shown, and the other compartments 130 can have notches 120″″ to allow opening. Or the seal 119 can be positioned along several or all of the compartments 130.

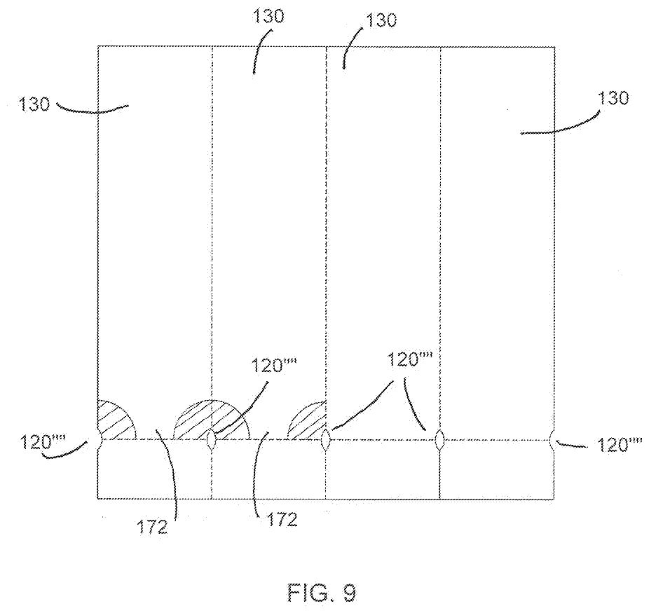

FIG. 9 shows the use of notches 120″″ for the compartments 130 and a spout 172 positioned at two of the compartments 130. FIGS. 10(a)-10(c) show additional non-limiting illustrative embodiments of the invention. As shown, the compartments 130 can be aligned in any suitable manner and with any suitable arrangement of divider walls 132 and/or notches or tear lines. As shown in FIG. 10(c), the notches 120 can have a design such as the shape of a heart. And the notches 120 can be positioned at the edge of the package or at the middle portion of the package. The notches 120 at the middle portion of the package can be close to the edge so that the user can tear through the reduced edge portion. Here, the notches 120 at the middle portion are aligned with the circular openings of the divider wall 132 so that the user can tear along the circular openings to separate the compartments. Of course, the notches at the middle portion can be aligned with an end or middle of a tear line.

Thus, a package is provided that is easy to open and can be a single use package that holds product such as health and/or beauty items. The overall package is flexible and relatively small and flat so it can easily carried and fit in a pocket, purse, drawer or the like. The plastic sheets are strong enough to prevent the product from leaking out of the compartments, but can also be tom open for use. The plastic is also flexible so that the package can be folded or bend when being carried. The package includes several separate compartments so that multiple products can be carried at one time. Multiple compartments can be opened at one time by aligning the opening mechanism to extend across those compartments. Or one or more of the compartments can have its own opening mechanism.

The description uses several geometric or relational terms, such as rectangular, parallel, and elongated. In addition, the description uses several directional or positioning terms and the like, such as top, bottom, left, right, up, down, inner, and outer. Those terms are merely for convenience to facilitate the description based on the embodiments shown in the figures. Those terms are not intended to limit the invention. Thus, it should be recognized that the invention can be described in other ways without those geometric, relational, directional or positioning terms. In addition, the geometric or relational terms may not be exact. For instance, walls may not be exactly perpendicular or parallel to one another but still be considered to be substantially perpendicular or parallel because of, for example, roughness of surfaces, tolerances allowed in manufacturing, etc. And, other suitable geometries and relationships can be provided without departing from the spirit and scope of the invention.

Within this specification, embodiments have been described in a way which enables a clear and concise specification to be written, but it is intended and will be appreciated that embodiments may be variously combined or separated without departing from spirit and scope of the invention. For example, it will be appreciated that all preferred features described herein are applicable to all aspects of the invention described herein. Thus, for example, a spout 172 (FIGS. 5, 9) is shown for a package where the opening mechanism is a notch 120 that is tom open to open the compartment 130. However, the spout 172 can be provided with any opening mechanism, such as the zipper 117 (FIG. 7) or seal 119 (FIG. 8).

Accordingly, a first embodiment of the invention is directed to a packaging having a first closed compartment, a second closed compartment, and an opening mechanism for opening the first and second closed compartments in a single operation by the user. In a second embodiment based on the first embodiment, the opening mechanism extends across the first and second closed compartments. In a third embodiment, a packaging is provided having multiple closed compartments and an opening mechanism for opening the multiple closed compartments in a single operation by the user. In a fourth embodiment based on the third embodiment, the packaging comprises a first plastic sheet and a second plastic sheet, the multiple compartments are formed between the first and second plastic sheets, and the opening mechanism comprises a notch in at least one side of the first and second plastic sheets. In a fifth embodiment, a method for forming a package is provided comprising providing a first plastic sheet and a second plastic sheet each having four sides, heat sealing three sides of the first and second sheets to one another, heat sealing divider walls to form a plurality of open compartments, filling product into each of the plurality of open compartments, and heat sealing the fourth side of the first and second sheets to close the plurality of open compartments to form a plurality of closed compartments. In a sixth embodiment based on any of the prior embodiments, all of the plurality of compartments relate to a single type of application. In a seventh embodiment based on any of the prior embodiments, each of the plurality of compartments retain a respective product. In an eighth embodiment based on the seventh embodiment, the respective products are for a same product type.

Each of the exemplary embodiments described above may be realized separately or in combination with other exemplary embodiments.

The foregoing description and drawings should be considered as illustrative only of the principles of the invention. The invention may be configured in a variety of shapes and sizes and is not intended to be limited by the preferred embodiment. For instance, although the opening mechanism is shown and described as being a notch 120, other suitable mechanisms can be provided, and the opening mechanism need not result in a tearing of the package. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

What is claimed is:

1. A package comprising:
a first closed compartment;
a second closed compartment attached with the first closed compartment;
a first opening mechanism extending at least partially across the first closed compartment for opening the first closed compartment in a single operation; and
a second opening mechanism extending at least partially across the second closed compartment for opening the second closed compartment in a single operation,
wherein at least one of the first opening mechanism and the second opening mechanism comprises a zipper.

2. A package comprising:
a body formed by a first plastic sheet and a second plastic sheet;
a first plurality of heat seals coupling the first plastic sheet and the second plastic sheet to define a first sealed elongated compartment formed between said first and second sheets; said first sealed elongated compartment having a first length;
a first product sealed within the first compartment;
a second plurality of heat seals coupling the first plastic sheet and the second plastic sheet to define a second sealed elongated compartment formed between said first and second sheets, said second sealed elongated compartment having a second length different than the first length, wherein said second sealed elongated compartment is directly attached to the first sealed elongated compartment and extends substantially parallel to the first sealed elongated compartment;
a second product sealed within the second compartment; and
an opening mechanism in each of said first and second sheets and extending at least partially across one end of each of the first and second compartments, whereby when the opening mechanism is operated the first and second compartments are respectively opened to release the first and second products.

3. The package of claim 2, further comprising at least one guide wall in said first compartment for guiding the first product out of the first compartment when opened.

4. The package of claim 2, further comprising a third plurality of heat seals coupling the first plastic sheet and the second plastic sheet to define a third sealed elongated compartment formed between said first and second sheets, wherein the first sealed elongated compartment is adjacent to, connected to, and shares a common heat seal with the second sealed elongated compartment, and the third sealed elongated compartment is adjacent to, connected to, and shares a common heat seal with each of the first and second sealed elongated compartments.

5. The package of claim 4, wherein the first, second and third sealed elongated compartments have a first, second and third longitudinal axis respectively, and the first and second longitudinal axes are substantially parallel and the third longitudinal axis is substantially perpendicular to the first and second longitudinal axes.

6. The package of claim 2 wherein each of the plurality of compartments contains a different product, and wherein the different products are components of a single type of application.

7. The package of claim 6, wherein the application is a cosmetic application.

8. The package of claim 6, wherein one of the different products is a tool for applying the other different product.

9. The package of claim 8, wherein the other different product is a cosmetic product and the tool is an applicator configured to apply the cosmetic product.

10. A method for forming a package comprising:
providing a first plastic sheet and a second plastic sheet each having four sides;
heat sealing three sides of the first and second sheets to one another;
heat sealing divider walls to form a plurality of open compartments;
filling product into each of the plurality of open compartments; and
heat sealing the forth side of the first and second sheets to close the plurality of open compartments to form a plurality of closed compartments.

11. The method of claim 10, wherein each of the plurality of compartments contains a different product, and wherein all of the different products are components of a single type of application.

12. The method of claim 10, wherein each of the plurality of compartments retains a respective product for a cosmetic application.

13. The method of claim 11, wherein at least one of the different products is a tool for applying at least one of the other different products.

14. The method of claim 13, wherein the at least one of the other different products is a cosmetic product and the tool is an applicator configured to apply the cosmetic product.
